(19) United States
(12) Patent Application Publication
(10) Pub. No.: US 2024/0390267 A1
(43) Pub. Date: Nov. 28, 2024

(54) DRUG DELIVERY DEVICE WITH CAPSULE LOCK

(71) Applicant: Biograil APS, Roskilde (DK)

(72) Inventors: Nikolaj Skak, Roskilde (DK); Karsten Lindhardt, Roskilde (DK); Kamille Majken Dumong Erichsen, Roskilde (DK)

(21) Appl. No.: 18/696,276

(22) PCT Filed: Oct. 28, 2022
(86) PCT No.: PCT/EP2022/080208

§ 371 Date: Mar. 27, 2024

(30) Foreign Application Priority Data
Oct. 28, 2021 (DK) PA202170526

(51) Int. Cl. A61K 9/00; A61J 3/07; A61K 9/48

(52) U.S. Cl. CPC A61K 9/0053; A61J 3/07; A61K 9/4808

(57) ABSTRACT

A drug delivery device comprising, a first body part, an attachment part attached to the first body part, a second body part, an aperture in the first body part and/or the second body part, an actuator mechanism configured to store energy and convert the stored energy to kinetic energy via movement of the first body part or the second body part, and a locking system configured to prevent the actuator mechanism from converting the stored energy to the kinetic energy.

The present disclosure relates to a drug delivery device and in particular to a drug delivery device for oral administration. The drug delivery device is advantageously configured for delivery of an active drug substance in the gastrointestinal tract including the stomach and/or intestines, such as the small intestines and/or the large intestines (colon).

### BACKGROUND

A number of for example low permeable and/or low water soluble active drug substances are currently delivered by i.e. subcutaneous, intradermal, intramuscular, rectal, vaginal or intravenous route. Oral administration has the potential for the widest patient acceptance and thus attempts to deliver low permeable and/or low water soluble active drug substances through the preferred oral route of administration has been tried but with limited success in particular due to lack of stability and limited absorption from the gastrointestinal tract.

Stability both relates to the stability of the active drug substance during manufacturing and storage of the delivery device, and to the stability of the active drug substance during the passage in the gastrointestinal tract before it become available for absorption.

Limited gastrointestinal absorption is due to the gastrointestinal wall barrier preventing active drug substance from being absorbed after oral dosing because of the low permeability of the active drug substance, which is for example due to pre-systemic metabolism, size and/or the charges and/or because of the water solubility of the active drug substance.

Multiple approaches to solve these stability and absorption challenges have been suggested, but an effective solution to the challenges remain unresolved.

### SUMMARY

Thus, there is an unmet need to provide a drug delivery device, which is capable of delivering drug substances for absorption in the gastrointestinal tissue. More generally, there remains a need for drug products and methods that enable enhanced drug delivery, when drug products are administered orally to patients.

Disclosed herein are examples of a drug delivery device. The drug delivery device includes a first body part. The drug delivery device includes an attachment part. The attachment part can be attached to the first body part. The drug delivery device includes a second body part. The drug delivery device includes an aperture. The aperture can be in the first body part and/or the second body part. The drug delivery device includes an actuator mechanism. The actuator mechanism can be configured to store energy. The actuator mechanism can be configured to convert the stored energy to kinetic energy. The actuator mechanism can be configured to convert the stored energy to kinetic energy via movement of the first body part or the second body part. The drug delivery device includes a locking system. The locking system can be configured to prevent the actuator mechanism from converting the stored energy to the kinetic energy. The locking system includes a locking member. The locking member can be configured to be releasably retained in the aperture. The locking member can be configured to prevent movement of the first body part with respect to the second body part. The locking system includes a biodegradable cover. The biodegradable cover can be configured to prevent translation of the locking member out of the aperture.

It is an advantage of the present disclosure that the drug delivery device secures stability of the active drug substance during passage in the gastrointestinal tract and facilitates effective absorption of the active drug substance from the gastrointestinal tract after oral administration.

Further, it is advantage of the present disclosure that the drug delivery device provides an active attachment of the drug delivery device to the gastro-internal wall, such as the stomach wall and/or intestine wall. Advantageously, the present disclosure can allow for more robust and reliable attachment of the drug delivery device. For example, the present disclosure does not require the use of a cover to protect the drug delivery device during locking mechanism dissolution. In one or more example drug delivery devices, there may be an integrated action of one or more of: locking mechanism dilution, capsule dissolution, and attachment part actuation.

Further, the present disclosure advantageously provides oral delivery of low permeable active drug substances in or at the gastro-internal tissue.

Further, it is an advantage of the present disclosure to improve the ease of manufacturing of a drug delivery device.

### BRIEF DESCRIPTION OF THE DRAWINGS

The above and other features and advantages of the present invention will become readily apparent to those skilled in the art by the following detailed description of exemplary embodiments thereof with reference to the attached drawings, in which:

FIG. 1 illustrates a diagram of an example drug delivery device having a locking member inserted into an aperture according to the disclosure,

FIG. 2 illustrates a diagram of an example drug delivery device having a locking member outside of an aperture according to the disclosure,

FIG. 3 illustrates a diagram of an example drug delivery device having an exploded biodegradable cover according to the disclosure,

FIG. 4 illustrates a diagram of an example drug delivery device having a biodegradable cover according to the disclosure, and

FIG. 5 illustrates a diagram of an example drug delivery device having a biodegradable cover according to the disclosure.

### DETAILED DESCRIPTION

Various exemplary embodiments and details are described hereinafter, with reference to the figures when relevant. It should be noted that the figures may or may not be drawn to scale and that elements of similar structures or functions are represented by like reference numerals throughout the figures. It should also be noted that the figures are only intended to facilitate the description of the embodiments and the functionalities associated therewith. They are not intended as an exhaustive description of the invention or as a limitation on the scope of the invention or the physical appearance of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

A drug delivery device is disclosed. The drug delivery device can include a first body part. The drug delivery device can include an attachment part. The attachment part can be attached to the first body part. The drug delivery device can include a second body part. The drug delivery device can include an aperture in the first body part and/or the second body part. The drug delivery device can include an aperture in the first body part. The drug delivery device can include an aperture in the second body part. The drug delivery device can include an actuator mechanism, such as an actuator. The actuator can be configured to store energy. The actuator can be configured to convert the stored energy to kinetic energy. The actuator can be configured to convert the stored energy to kinetic energy via movement of the first body part or the second body part. The drug delivery device can include a locking system. The locking system can be configured to prevent the actuator mechanism from converting the stored energy to the kinetic energy. The locking system can include a locking member. The locking member can be configured to be releasably retained in the aperture. The locking member can be configured to prevent movement of the first body part with respect to the second body part. The locking system can include a biodegradable cover. The biodegradable cover can be configured to prevent translation of the locking member out of the aperture.

The drug delivery device may have a size and geometry designed to fit into a pharmaceutical composition for oral administration.

The drug delivery device/pharmaceutical composition may be configured to be taken into the body via the oral orifice. Thus, the outer dimensions of the drug delivery device/pharmaceutical composition may be small enough for a user to swallow. The drug delivery device may be adapted to carry a drug substance into the body of the user, via the digestive system, so that the drug delivery device may e.g. travel from the mouth of the user into the stomach, via the oesophagus. The drug delivery device may further travel into the intestines from the stomach, and may optionally travel into the bowels and out through the rectum.

The drug delivery device may be configured to deliver the drug in any part of the digestive system of the user, where in one example it may be configured to deliver a drug substance into the stomach of the user. In another example, the drug delivery device may be adapted to initiate the drug delivery when the device has passed the stomach and has entered the intestine of the user. In other words, the drug delivery device may be configured to attach to a wall of the stomach or a wall of the intestines, e.g. depending on the desired release position of the active drug substance.

The attachment part(s) of the drug delivery device may be configured to interact with the inner surface linings of the gastrointestinal tract, so that the drug delivery device may e.g. be attached to the inner surface (mucous membrane) of the stomach, or alternatively to the mucous membrane of the intestines. The attachment part(s) may be configured to interact with the mucous membranes, e.g. in order to fix or attach the drug delivery device, e.g. for a period of time, inside the body of the user. By attaching the drug delivery device, it will allow a drug substance to be delivered into a part of the digestive system, in order to provide a drug substance to the body of the user. The attachment part(s) may be configured to interact with the mucous membranes, e.g. in order to inject drug substance into the gastrointestinal tract wall.

The drug delivery device has a central axis optionally extending from a first end to a second end of the drug delivery device. The drug delivery device may have a length (e.g. largest extension from first end to second end along central axis), in the range from 3 mm to 35 mm, such as in the range from 5 mm to 26 mm. The drug delivery device may be elongated.

The drug delivery device may have a width and/or height (e.g. largest extensions along width axis and height axis, respectively) in the range from 1 mm to 20 mm. Height and width are the largest extensions of the drug delivery device perpendicular to the central axis.

In one or more exemplary drug delivery devices, the dimensions of the drug delivery device, at least in an initial state or first state prior to actuation of the first attachment part and/or the second attachment part, may be represented by a length (largest extension along central axis), a width (largest extension along width axis perpendicular to the central axis) and a height (largest extension along height axis perpendicular to the central axis and the width axis). The height of the drug delivery device may be in the range from 1 mm to 15 mm. The width of the drug delivery device may be in the range from 1 mm to 15 mm.

In one or more exemplary drug delivery devices, the drug delivery device may be constructed in a way that secures the drug delivery part to deliver a payload or active drug substance into the internal tissue or internal surface for distribution of the active drug substance in the subject through the blood vessels.

Advantageously, the drug delivery device may be attached, and may deliver the active drug substance, to a particular location in a patient's intestinal wall. Of course, the delivery device may be attached, and may deliver the active drug substance, to other places as well. In one or more exemplary drug delivery devices, the drug delivery device, such as the spike, may penetrate the muscularis mucosa. In one or more exemplary drug delivery devices, the drug delivery device, such as the spike, may not penetrate the muscularis externa. In one or more exemplary drug delivery devices, the spike may be positioned in the submucosa. In one or more exemplary drug delivery devices, the spike may be positioned in the submucosa parallel to the gut wall.

The drug delivery device comprises a first body part. The first body part may be a two-part body part, i.e. the first body part may comprise a first primary body part and a first secondary body part. The first body part has an outer surface. A first primary recess and/or a first secondary recess may be formed in the outer surface of the first body part.

The drug delivery device optionally comprises a shell having a first shell part. An outer surface of the first body part may constitute at least a part of the first shell part.

The drug delivery device comprises a first attachment part. The first attachment part may comprise a first base part and/or a first needle, e.g. a spike. The first attachment part has a first proximal end and a first distal end. The first attachment part, such as the first needle or spike, optionally has or extends along a first attachment axis. A first tip of the first needle forms the first distal end. In other words, the first distal end is a first tip of the first needle. The first base may be arranged at or constitute the first proximal end of the first attachment part. The first needle may have a length in the range from 1 mm to 15 mm such, as in the range from 3 mm to 10 mm. Thereby sufficient penetration into the internal tissue may be provided for while at the same time reducing the risk of damaging the internal tissue. The first distal end of the first attachment part may be provided with a tip configured to penetrate a biological tissue. The first distal end of the first attachment part may be provided with a gripping part configured to grip a biological tissue.

The first needle may have a cross-sectional diameter in the range from 0.1 mm to 5 mm, such as in the range from 0.5 mm to 2.0 mm.

The first needle may be straight and/or curved. The first needle may comprise a first primary section that is straight. The first needle may comprise a first secondary section, e.g. between the first primary section and the first distal end or between the first base and the first primary section. The first secondary section may be curved.

The first needle may include two or more straight portions formed at an angle. For example, the first needle may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion. The joint angle may be an acute angle, an obtuse angle, or a right angle. The angle may be, for example, 10, 20, 30, 40, 50, 60, 70, 80, 90, 100, 120, 130 140, 150, 160, or 170 degrees. This can advantageously allow for different angles of attach when the first needle interacts with inner surface linings. This may allow for improved attachment of the drug delivery device, while helping to reduce or avoid tissue damage. Further, the joint may be flexible. Alternatively, the joint may not be flexible.

The joint may be located at a center, or generally at a center, of a length of the first needle. Alternatively, the joint may be located 40, 45, 55, 60, or 65% up a length of the first needle from the proximal end.

In one or more exemplary first attachment parts, the first needle may have three, four, or five different portions at different angles, each connected by a joint. In some iterations, any or all of the different portions may be straight or curved. Each joint may be flexible or not flexible.

The attachment parts of the drug delivery device may be seen as any kind of attachment parts that may be capable of attaching the drug delivery device to a biological tissue, such as a stomach wall, a wall of the bowels and/or intestines of a human or animal body. The attachment parts may be adapted to extend in a direction away from the central axis of the drug delivery device, and/or a central axis of the first attachment part. This may mean that the attachment part(s), e.g. at least in an activated state or second state of the drug delivery device, may extend in a direction away from a peripheral surface (in radial direction) of the first body part and/or the second body part, so that the attachment part extends farther in a radial direction than the peripheral or outer surface of the body part.

The first attachment part may be fixed or rotationally attached to the first body part.

In one or more exemplary drug delivery devices, the drug delivery device comprises a second body part. The second body part may be a two-part body part, i.e. the second body part may comprise a second primary body part and a second secondary body part. The second attachment part is optionally attached to the second body part. The second attachment part may be fixed or rotationally attached to the second body part. The second body part has an outer surface. A second primary recess and/or a second secondary recess may be formed in the outer surface of the second body part.

In one or more exemplary drug delivery devices, the actuator mechanism is configured to store energy. For example, the actuator mechanism can be configured to store one or more of: potential energy, chemical potential energy, including effervescent energy, spring energy, elastic potential energy, electrical potential energy, gravitational potential energy, thermal energy, and compression energy such as from compressed air and/or expanding polymers. The particular type of stored energy is not limiting.

The actuator mechanism can be configured to convert, such as release, expel, unload, change, the stored energy to kinetic energy. For example, the actuator mechanism can be configured to convert the stored energy to kinetic energy via movement of the first body part or the second body part. The actuator mechanism can be configured to convert the stored energy to kinetic energy via movement of the first body part and/or the second body part with respect to one another. The actuator mechanism can be configured to convert the stored energy to kinetic energy via rotational movement of the first body part and/or the second body part with respect to one another. The actuator mechanism can be configured to convert the stored energy to kinetic energy via translational movement of the first body part and/or the second body part with respect to one another. For example, the first body part may be translated away from the second body part, which can force the attachment part(s) into tissue. The actuator mechanism can be configured to convert the stored energy to kinetic energy via translational movement and rotational movement of the first body part and/or the second body part with respect to one another.

In one or more example drug delivery systems, translational movement, such as longitudinal movement, of the first body part and the second body part can be configured to convert the stored energy to kinetic energy.

In one or more example drug delivery systems, release of the second body part from the first body part can be configured to convert the stored energy to kinetic energy.

In one or more example drug delivery devices, the actuator mechanism can be configured to convert the stored energy to the kinetic energy by rotating at least one of the first body part or the second body part with respect to one another.

In one or more example drug delivery devices, the actuator mechanism can be configured to rotate the first body part and/or the second body part with respect to each other. In one or more exemplary drug delivery devices, the actuator mechanism is configured to rotate the first body part in relation to the second body part about a primary axis of the drug delivery device. The primary axis may be parallel to and/or coinciding with the central axis. Alternatively, or in conjunction, the actuator mechanism can be configured to non-rotationally translate the first body part in relation to the second body part.

In one or more exemplary drug delivery devices, the first body part is configured to rotate in a first direction and/or the second body part is configured to rotate in a second direction opposite to the first direction. For example, this may occur due to the conversion of the stored energy to kinetic energy.

The drug delivery device may comprise a frame part, where different parts, such as the first body part and/or the second body part are attached, e.g. fixed or rotatably attached to the frame part. In one or more exemplary drug delivery devices, the actuator mechanism, or parts thereof, may be attached to the frame part. Thereby, separate rotation of the first body part and the second body part in relation to the frame part may be provided for

The rotational connection between the first body part and the second body part allows the first body part to rotate relative to the second body part, without the two parts separating from each other before the attachment part(s) interact with the internal tissue, such as mucous membranes. Such a connection may be obtained in a plurality of ways, where in one example the first body part has a plug connection and the second body part has a socket connection, where this plug and socket configuration allows the first body part to rotate relative to the second body part. A second example could be to provide an axle that may be coaxial with the central axis and/or the primary axis, where the first body part and the second body part are configured to receive the axle, and a stopping device is arranged at first and second ends of the axle, on each side of the combined first and second body part, preventing the first body part and the second body part to slide in a longitudinal direction along the axle. The axle may be integrated in the first body part or in the second body part.

If an axle were used, the axle can be made of any number of different materials. For example, the axle can be made of metals and/or alloys and/or polymers and/or composites and/or composites and/or combinations thereof.

The first and/or the second body part may be arranged to rotate freely relative to each other, e.g. at least in the second state, and thereby allowing the attachment parts to rotate relative to each other. Thus, the attachment parts may be adapted to come into contact and/or penetrate tissue of the gastrointestinal tract. The rotation of the body parts relative to each other using a resilient force may move the attachment parts in such a way that they are capable of e.g. penetrating or pinching the mucous membrane in order to fix the drug delivery device at a location in the gastrointestinal tract, such as the stomach or intestines. The penetrating and/or pinching force may come from the actuator mechanism/resilient part, where the resilient part may be adapted to store a resilient force that is capable of forcing the attachment parts towards each other when the resilient force of the resilient part has been at least partly unleashed. The resilient part may e.g. be in the form of a spring or spring element, for example a torsional spring or a power spring, where the spring may be wound up to store mechanical energy, where the mechanical energy may be transmitted to the first and/or the second body part. When the mechanical energy is released, the first body part may rotate relative to the second body part, and where the mechanical energy may be transferred into the attachment parts via the body parts.

Within the context of the present description the term “rotational force” may be seen as Torque, moment, moment of force, rotational force or “turning effect”. Another definition of the term “rotational force” may be the product of the magnitude of the force and the perpendicular distance of the line of action of force from the axis of rotation. The rotational force may be seen as the force which is transferred from the resilient part to the attachment members of the drug delivery device via the body parts.

The conversion to kinetic energy, such as the rotational force may be defined as being large enough to penetrate into the gastrointestinal tissue. When the rotational force is applied to both the first and the second body part, the first attachment member may come into contact with the surface to be attached to, and where the rotational force applied to the second body part may cause the second attachment part to come into contact with the same surface, where the first attachment part provides a force, while the second attachment part provides a counter force to the first attachment part, so that the force is applied in such a manner that the first attachment part is forced in a direction towards the second attachment part, or vice versa. Translational force can be used as well.

In one or more exemplary drug delivery devices, a distance between the first attachment axis of the first attachment part and the primary axis, e.g. at least in an activated state or second state of the drug delivery device and optionally in an initial state of the drug delivery device, is larger than 0.5 mm.

In one or more exemplary drug delivery devices, a distance between the second attachment axis of the second attachment part and the primary axis, e.g. at least in an activated state or second state of the drug delivery device and optionally in an initial state of the drug delivery device, is larger than 0.5 mm.

In one or more exemplary drug delivery devices, the first attachment part is rotationally attached to the first body part, e.g. via a first joint connection having a first rotation axis. In other words, the first attachment part is optionally configured to rotate about a first rotation axis, e.g. in relation to the first body part. The first rotation axis may be parallel to the central axis and/or the primary axis. The first rotation axis may form a first angle with the central axis and/or the primary axis. The first angle may be less than 15°. The first angle may be in the range from 75° to 105°, such as 90°+5° or 90°.

In one or more exemplary drug delivery devices, the first body part may define a first body recess (e.g., cavity, slot, hole) extending to an outer surface of the first body part. The first body recess may be formed by solid walls on all sides except an outermost surface which is open. The first attachment part may be rotationally connected within the first body recess along a first attachment part axis. The first attachment part axis may be, for example, a pin (e.g., arm, support). The first attachment part axis may be parallel to the central axis and/or the primary axis. The first attachment part axis may be angled with respect to the central axis and/or the primary axis. Accordingly, the first attachment part may be configured to rotate within the recess along the first attachment part axis. Further, rotation of the first attachment part may be stopped at end surfaces of the recess.

The first body recess may extend along a portion of the outer surface of the first body. The first body recess may extend fully along an outer circumference of the first body. The first body recess may extend around 10, 15, 20, 25, 30, 35, 40, 45, 50, 55, 60, 65, 70, 75, 80, 85, 90, 95, or 100% of an outer circumference of the first body. The first body recess may extend around greater than 10, 15, 20, 25, 30, 35, 40, 45, 50, 55, 60, 65, 70, 75, 80, 85, 90, or 95% of an outer circumference of the first body. The first body recess may extend around less than 10, 15, 20, 25, 30, 35, 40, 45, 50, 55, 60, 65, 70, 75, 80, 85, 90, 95, or 100% of an outer circumference of the first body. The first body may optionally contain more than one first body recess, for example of a plurality of first attachment parts are used on the first body. If more than one first body recess is used, they may be spaced longitudinally apart and/or circumferentially apart.

The first body recess may extend from an outer surface toward the central axis through 5, 10, 15, 20, 25, 30, 35, or 40% of the drug delivery device. The first body recess may extend from an outer surface toward the central axis through greater than 5, 10, 15, 20, 25, 30, 35, or 40% of the drug delivery device. The first body recess may extend from an outer surface toward the central axis through less than 5, 10, 15, 20, 25, 30, 35, or 40% of the drug delivery device.

In one or more exemplary drug delivery devices, the first body recess may extend circumferentially, or partially circumferentially around the first body with the central axis being the longitudinal direction. The first body recess may extend perpendicularly with respect to the central axis and/or the primary access (e.g., may extend along a cross section of the drug delivery device perpendicular to the central axis and/or the primary access). The first body recess may have any number of shapes. For example, the first body recess can be a portion of a circle, such as a half circle. The first body recess can be a triangle. The first body recess can be a sector of a circle. The first body recess can be a curved edge connected by two straight edge. The first body recess can be two curved edges connected to each other by two straight edges.

Thus, the first attachment part may rotate on the first attachment part axis in order to move perpendicular to the central axis and/or the primary axis. In certain embodiments, the first attachment part may rotate at an angle between perpendicular and parallel with respect to the central axis and/or the primary axis.

In one or more exemplary drug delivery devices, when the first body part and/or the second body part rotate with respect to one another, the first attachment part and/or the second attachment part can rotate out of their respective recesses (e.g., first body recess and second body recess) due to the rotation of the first body part and/or the second body part. The continued rotation of the first body part and/or the second body part then causes the first attachment part and/or the second attachment part to pierce tissue to hold the drug delivery device in place.

In one or more exemplary drug delivery devices, the first attachment part extends, e.g. at least in an activated state of the drug delivery device and optionally in an initial state of the drug delivery device, in a direction away from the first body part. In other words, the first needle may extend, e.g. at least in an activated state of the drug delivery device and optionally in an initial state, from an outer surface of the first body part. Formulated differently, the first attachment axis may, e.g. at least in an activated state of the drug delivery device and optionally in an initial state of the drug delivery device form an angle of at least 45° with the central axis and/or the primary axis. An attachment part extending in a direction is to be understood as the direction from proximal end of attachment part/needle part to distal end of attachment part along the attachment axis of the attachment part.

The first attachment part may in a first state of the drug delivery device extend in a first primary direction and in a second state of the drug delivery device extend in a first secondary direction. The first primary direction and the first secondary direction may form an angle of at least 30°. The first primary direction may be parallel or substantially parallel to the central axis. The first primary direction may form an angle less than 60° with the central axis. The first secondary direction may form an angle of at least 60° such as about 90° with the central axis. The first secondary direction may be perpendicular to the central axis.

The first distal end of the first attachment part may be configured to move or be moved from a first primary position in a first state of the drug delivery device to a first secondary position in the second state.

The drug delivery device comprises a second attachment part. The second attachment part may comprise a second base part and/or a second needle, e.g. spike. The second attachment part has a second proximal end and a second distal end. The second attachment part, such as the second needle, optionally has or extends along a second attachment axis. A second tip of the second needle forms the second distal end. In other words, the second distal end is a second tip of the second needle. The second base may be arranged at or constitute the second proximal end of the second attachment part. The second needle may have a length in the range from 1 mm to 15 mm such, as in the range from 3 mm to 10 mm. Thereby sufficient penetration into the internal tissue may be provided for while at the same time reducing the risk of damaging the internal tissue. The second distal end of the second attachment part may be provided with a tip configured to penetrate a biological tissue. The second distal end of the second attachment part may be provided with a gripping part configured to grip a biological tissue.

The second needle may have a cross-sectional diameter in the range from 0.1 mm to 5 mm, such as in the range from 0.5 mm to 2.0 mm.

The second needle may be straight and/or curved. The second needle may comprise a second primary section that is straight. The second needle may comprise a second secondary section, e.g. between the second primary section and the second distal end or between the second base and the second primary section. The second secondary section may be curved.

The second needle may include two or more straight portions formed at an angle. For example, the second needle may have a proximal portion that extends at a first angle from a connection point to the drug delivery device and a distal portion that extends at a second angle from a connection point to the drug delivery device. The first angle and the second angle may be different. The proximal portion may connect to the distal portion at a joint (e.g., bend, connection, angle) and have a joint angle between the proximal portion and the distal portion. The joint angle may be an acute angle, an obtuse angle, or a right angle. The angle may be, for example, 10, 20, 30, 40, 50, 60, 70, 80, 90, 100, 120, 130 140, 150, 160, or 170 degrees. This can advantageously allow for different angles of attach when the second needle interacts with inner surface linings. This may allow for improved attachment of the drug delivery device, while helping to reduce or avoid tissue damage. Further, the joint may be flexible. Alternatively, the joint may not be flexible.

The joint may be located at a center, or generally at a center, of a length of the second needle. Alternatively, the joint may be located 40, 45, 55, 60, or 65% up a length of the second needle from the proximal end.

In one or more exemplary second attachment parts, the second needle may have three, four, or five different portions at different angles, each connected by a joint. In some iterations, any or all of the different portions may be straight or curved. Each joint may be flexible or not flexible.

In one or more exemplary drug delivery devices, both the first needle and the second needle include a joint. However, only one of the first needle and the second needle may include a joint with the other being straight and/or curved. If both the first needle and the second needle include a joint, the first distal tip and the second distal tip may be angled towards one another in order to facilitate attachment when the first body part and the second body part rotate with respect to one another.

In one or more exemplary drug delivery devices, the second attachment part is rotationally attached to the second body part, e.g. via a second joint connection having a second rotation axis. In other words, the second attachment part is optionally configured to rotate about a second rotation axis, e.g. in relation to the second body part. The second rotation axis may be parallel to the central axis and/or the primary axis. The second rotation axis may form a second angle with the central axis and/or the primary axis. The second angle may be less than 15°. The second angle may be in the range from 75° to 105°, such as 90°±5° or 90°.

In one or more exemplary drug delivery devices, the second body part may define a second body recess (e.g., cavity, slot, hole) extending to an outer surface of the second body part. The second body recess may be formed by solid walls on all sides except an outermost surface which is open. The second attachment part may be rotationally connected within the second body recess along a second attachment part axis. The second attachment part axis may be, for example, a pin (e.g., arm, support). The second attachment part axis may be parallel to the central axis and/or the primary axis. The second attachment part axis may be angled with respect to the central axis and/or the primary axis. Accordingly, the second attachment part can be configured to rotate within the recess along the second attachment part axis. Further, rotation of the second attachment part may be stopped at end surfaces of the second body recess.

The second body recess may extend along a portion of the outer surface of the second body. The second body recess may extend fully along an outer circumference of the second body. The second body recess may extend around 10, 15, 20, 25, 30, 35, 40, 45, 50, 55, 60, 65, 70, 75, 80, 85, 90, 95, or 100% of an outer circumference of the second body. The second body recess may extend around greater than 10, 15, 20, 25, 30, 35, 40, 45, 50, 55, 60, 65, 70, 75, 80, 85, 90, or 95% of an outer circumference of the second body. The second body recess may extend around less than 10, 15, 20, 25, 30, 35, 40, 45, 50, 55, 60, 65, 70, 75, 80, 85, 90, 95, or 100% of an outer circumference of the second body. The second body may optionally contain more than one second body recess, for example of a plurality of second attachment parts are used on the second body. If more than one second body recess is used, they may be spaced longitudinally apart and/or circumferentially apart.

The second body recess may extend from an outer surface toward the central axis through 5, 10, 15, 20, 25, 30, 35, or 40% of the drug delivery device. The second body recess may extend from an outer surface toward the central axis through greater than 5, 10, 15, 20, 25, 30, 35, or 40% of the drug delivery device. The second body recess may extend from an outer surface toward the central axis through less than 5, 10, 15, 20, 25, 30, 35, or 40% of the drug delivery device.

In one or more exemplary drug delivery devices, the second body recess may extend circumferentially, or partially circumferentially around the second body with the central axis being the longitudinal direction. The second body recess may extend perpendicularly with respect to the central axis and/or the primary access (e.g., may extend along a cross section of the drug delivery device perpendicular to the central axis and/or the primary access). The second body recess may have any number of shapes. For example, the second body recess can be a portion of a circle, such as a half circle. The second body recess can be a triangle. The second body recess can be a sector of a circle. The second body recess can be a curved edge connected by two straight edge. The second body recess can be two curved edges connected to each other by two straight edges.

Thus, the second attachment part may rotate on the second attachment part axis in order to move perpendicular to the central axis and/or the primary axis. In certain embodiments, the second attachment part may rotate at an angle between perpendicular and parallel with respect to the central axis and/or the primary axis.

In one or more exemplary drug delivery devices, when the first body part and/or the second body part rotate with respect to one another, the first attachment part and/or the second attachment part can rotate out of their respective recesses (e.g., first body recess and second body recess) due to the rotation of the first body part and/or the second body part. The continued rotation of the first body part and/or the second body part then causes the first attachment part and/or the second attachment part to pierce tissue to hold the drug delivery device in place.

In one or more exemplary drug delivery devices, the second attachment part extends, e.g. at least in an activated state of the drug delivery device and optionally in an initial state of the drug delivery device, in a direction optionally away from the second body part. In other words, the second needle may extend, e.g. at least in an activated state of the drug delivery device and optionally in an initial state, from an outer surface of the second body part. Formulated differently, the second attachment axis may, e.g. at least in an activated state of the drug delivery device and optionally in an initial state of the drug delivery device form an angle of at least 45° with the central axis and/or the primary axis.

The second attachment part may in a first state of the drug delivery device extend in a second primary direction and in a second state of the drug delivery device extend in a second secondary direction. The second primary direction and the second secondary direction may form an angle of at least 30°. The second primary direction may be parallel or substantially parallel to the central axis. The second primary direction may form an angle less than 60° with the central axis. The second secondary direction may form an angle of at least 60°, such as about 90° with the central axis. The second secondary direction may be perpendicular to the central axis.

The second distal end of the second attachment part may be configured to move or be moved from a second primary position in a first state of the drug delivery device to a second secondary position in the second state.

The drug delivery device comprises an actuator mechanism. The actuator mechanism can be configured to store energy. The actuator mechanism can be configured to convert the stored energy to kinetic energy. For example, the actuator mechanism is configured to move the first attachment part in relation to the second attachment part, such as configured to move the first distal end towards and/or away from the second distal end, e.g. at least during a part of a rotation and/or a translation, such as in a first rotation and optionally in a second rotation. To move the first distal end towards the second distal end may be understood as reducing a distance between the first distal end and the second distal end. To move the first distal end towards the second distal end may be understood as reducing an angle between the first attachment axis and the second attachment axis, such as reducing an angle between a first secondary direction of the first attachment part and a second secondary direction of the second attachment part. In one or more exemplary drug delivery devices, the actuator mechanism is configured convert stored energy to kinetic energy to move the first distal end towards the second distal end by rotating, e.g. in a second state of the drug delivery device, the first body part in relation to the second body part and/or vice versa. The actuator mechanism may be configured to convert stored energy to kinetic energy to rotate the first body part at least 90°, such as at least 450°, at least 810°, at least 1170°, at least 1530°, or even at least 1890° in relation to the second body part about the primary axis. The actuator mechanism may be configured to convert stored energy to kinetic energy to rotate the first body part in relation to the second body part about the primary axis in a stepwise manner. In other words, to rotate the first body part in relation to the second body part about the primary axis may comprise a plurality of rotations including a first rotation and a second rotation, e.g. a first rotation followed by a first time period with reduced or no rotation followed by a second rotation. A first rotation followed by a second rotation after a first time period may increase the possibility of the drug delivery device attaching to the biological tissue. The first time period or in general time periods between rotations allows the drug delivery to move to other positions in the gastrointestinal tract. In other words, if the drug delivery does not attach to the biological tissue during a first rotation, further rotations increase the chance of attachment to the internal tissue. The first rotation may be at least 90°, and the second rotation may be at least 180°. The plurality of rotations may comprise a third rotation. The third rotation may be at least 180°.

In one or more exemplary drug delivery devices, a movement of the first distal end towards the second distal end may be preceded by and/or followed by a movement of the first distal end away from the second distal end. In other words, a movement of the first distal end towards the second distal end may be prior to and/or after movement of the first distal end away from the second distal end. For example, a first rotation may comprise moving the first distal end towards the second distal end and/or moving the first distal end away from the second distal end. For example, a second rotation may comprise moving the first distal end towards the second distal end and/or moving the first distal end away from the second distal end. For example, a third rotation may comprise moving the first distal end towards the second distal end and/or moving the first distal end away from the second distal end.

The actuator mechanism optionally comprises a resilient part such as a spring element configured to apply force to the first body part and/or the second body part. The spring element may be configured to store energy, such as elastic potential energy. The resilient part may comprise a first part, such as a first end, connected to the first body part. The resilient part may comprise a second part, such as a second end, connected to the second body part.

In one or more exemplary drug delivery devices, the actuator mechanism optionally comprises a swelling media, i.e. a media increasing its volume, e.g. upon contact with a fluid, e.g. in order to provide rotation of parts in relation to each other. In one or more exemplary drug delivery devices, a swelling medial provides rotation of the first attachment part in relation to the first body part and/or provides rotation of the second attachment part in relation to the second body part. In one or more exemplary drug delivery devices, a swelling media provides rotation of the first body part in relation to the second body part. The swelling media can similarly be configured to store energy and convert the stored energy to kinetic energy.

The actuator mechanism, such as the resilient part, may be configured to rotate the first attachment part about the first rotation axis in relation to the first body part, for example during the conversion of the stored energy to the kinetic energy.

The actuator mechanism, such as the resilient part, may be configured to rotate the second attachment part about the second rotation axis in relation to the second body part, for example during the conversion of the stored energy to the kinetic energy.

In one or more exemplary drug delivery devices, the drug delivery device comprises a first compartment, the drug delivery device being configured to deliver an active drug substance from the first compartment to the surroundings of the drug delivery device. The first compartment may be arranged in the first attachment part, such as in the first needle, e.g. within a distance of 8 mm, such as within 5 mm, from the first distal end. The first attachment part, such as the first needle may have one or more openings providing access to the first compartment. In one or more exemplary drug delivery devices, the first compartment is formed as a through-going bore in the first needle.

The first compartment may be arranged in any part of the drug delivery device, such as in the form of a cavity inside a volume of the first body part, the second body part or both of the first body part and the second body part. Additionally, or alternatively, the first compartment may be a compartment which is inside the first attachment part, where the penetration of the first attachment part into a biological tissue may release a drug substance in the first compartment into the biological tissue. Additionally or alternatively, the first compartment may be compartment that is in the form of a depression or opening or spike or hollow spike on the outer surface of the first and/or the second body part, where the drug delivery device may be adapted to release the drug substance inside the organ of the body which the drug delivery device is adapted to pass through.

In one or more exemplary drug delivery devices, the first compartment may be open from an inner volume of the drug delivery device and towards an outer part of the drug delivery device. In one or more examples, the first compartment may be inside the first body part, and where the first compartment is in fluid connection with the first attachment part, so that when the first distal end of the first attachment part has penetrated the biological tissue, the drug substance may be released from the first compartment and into the biological tissue via the first attachment part. This may e.g. be where the first attachment part is a tubular part, which has a first distal end in fluid communication with the first compartment of the drug delivery device.

In one or more exemplary drug delivery devices, the drug delivery device comprises a second compartment, the drug delivery device being configured to deliver an active drug substance from the second compartment to the surroundings of the drug delivery device. The second compartment may be arranged in the first attachment part or in the second attachment part, such as in the second needle, e.g. within a distance of 8 mm, such as within 5 mm, from the second distal end. The second attachment part, such as the second needle may have one or more openings providing access to the second compartment. In one or more exemplary drug delivery devices, the second compartment is formed as a through-going bore in the first needle or in the second needle.

In one or more exemplary drug delivery devices, the first attachment part and the second attachment part form an angle when the first distal end and the second distal end are in a plane that includes the primary axis. In other words, the first attachment axis and the second attachment may form an angle, e.g. larger than 5°, such as in the range from 10° to 75°, when the first distal end and the second distal end are in a plane that includes the primary axis.

In one or more exemplary drug delivery devices, the drug delivery device has a first state, also denoted initial state, where the first body part and the second body part are rotationally stationary relative to each other and a second state, also denoted activated state, where the first body part and the second body part are rotationally mobile relative to each other, e.g. can rotate about the primary axis of the drug delivery device. In other words, the first body part may be locked, e.g. prevented from rotating, in relation to the second body part. The first state may e.g. be an initial state or introduction state, where the drug delivery device is adapted to be introduced into the body, and where the first body part and the second body part are stationary relative to each other. In the first state the resilient part may have a predefined amount of stored energy, where the energy level is stationary in the resilient part while the body parts are stationary. For example, the first state may occur when the actuator mechanism stores energy. The second state may occur when the actuator mechanism is converting the stored energy to kinetic energy.

In one or more exemplary drug delivery devices, the drug delivery device has a first state where the resilient part has a constant resilient force load, such as a stored energy, and a second state where the resilient part at least partly releases the resilient force load, such as at least partially converting the stored energy to kinetic energy. In other words, the resilient part may be biased or preloaded in the first state of the drug delivery, such as storing energy, and upon release, e.g. by release of a locking system, (i.e. the drug delivery device being in the second state) the force from the resilient part may effect a rotation of the first body part in relation to the second body part, i.e. including a movement of the first distal end towards the second distal, such as a conversion of the stored energy to kinetic energy.

In one or more exemplary drug delivery devices, the actuator mechanism is configured to move the first distal end from a first primary position, e.g. in first state of the drug delivery device, with a first primary radial distance from a central axis of the delivery device to a first secondary position, e.g. in second state of the drug delivery device, with a first secondary radial distance from the central axis and/or primary axis, wherein the first secondary radial distance is larger than the first primary radial distance. Thus, the first distal end of the first attachment may be in a first primary position when the drug delivery device is in the first state and/or the first distal end of the first attachment part may be in a first secondary position when the drug delivery device is in the second state.

The first primary radial distance may be less than 10 mm, such as less than 8 mm or even less than 5 mm. The first secondary radial distance may be larger than the first primary radial distance. The first secondary radial distance may be larger than 5 mm, such as larger than 6 mm, or larger than 8 mm. In one or more exemplary drug delivery devices, the first secondary radial distance is in the range from 6 mm to 15 mm.

In one or more exemplary drug delivery devices, the first attachment part, such as a part of the first needle and/or the first distal end, may, in the first state be arranged or at least partly arranged within a first primary recess of the first body part. In the first state, the first distal end may be arranged inside the first body part.

In one or more exemplary drug delivery devices, the first attachment part, such as a part of the first needle and/or the first distal end, may, in the second state be arranged or at least partly arranged outside the first primary recess of the first body part.

In one or more exemplary drug delivery devices, the first attachment part, such as a part of the first needle and/or the first distal end, may, in the first state be arranged within a second primary recess of the second body part. Thereby, the first attachment part may be configured to lock the first body part in relation to the second body part in the first state of the drug delivery device.

In one or more exemplary drug delivery devices, the first attachment part, such as a part of the first needle and/or the first distal end, may, in the second state be arranged outside the second body part and/or at least outside the second primary recess of the second body part.

In one or more exemplary drug delivery devices, the actuator mechanism is configured to move the second distal end from a second primary position, e.g. in first state of the drug delivery device, with a second primary radial distance from a central axis of the delivery device to a second secondary position, e.g. in second state of the drug delivery device, with a second secondary radial distance from the central axis and/or primary axis, wherein the second secondary radial distance is larger than the second primary radial distance. Thus, the second distal end of the second attachment may be in a second primary position when the drug delivery device is in the first state and/or the second distal end of the second attachment part may be in a second secondary position when the drug delivery device is in the second state.

The second primary radial distance may be less than 10 mm, such as less than 8 mm or even less than 5 mm. The second secondary radial distance may be larger than the second primary radial distance. The second secondary radial distance may be larger than 5 mm, such as larger than 6 mm, or larger than 8 mm. In one or more exemplary drug delivery devices, the second secondary radial distance is in the range from 6 mm to 15 mm.

In one or more exemplary drug delivery devices, the second attachment part, such as a part of the second needle and/or the second distal end, may, in the first state be arranged or at least partly arranged within a first secondary recess of the first body part. Thereby, the second attachment part may be configured to lock the first body part in relation to the second body part in the first state of the drug delivery device. In the first state, the second distal end may be arranged inside the second body part.

In one or more exemplary drug delivery devices, the second attachment part, such as a part of the second needle and/or the second distal end, may, in the second state be arranged or at least partly arranged outside the first body part and/or at least outside the first secondary recess of the first body part.

In one or more exemplary drug delivery devices, the second attachment part, such as a part of the second needle and/or the second distal end, may, in the first state be arranged within a second secondary recess of the second body part.

In one or more exemplary drug delivery devices, the second attachment part, such as a part of the second needle and/or the second distal end, may, in the second state be arranged outside the second secondary recess of the second body part.

In one or more exemplary drug delivery devices, the actuator mechanism is configured to move, e.g. by rotation about a first rotation axis of the first attachment part (first base part), the first distal end from a first primary angular position of first primary position to a first secondary angular position of first secondary position in relation to a first proximal end of the first attachment part. An angle between the first primary angular position and the first secondary angular position may be larger than 10°, such as larger than 45° or larger than 60°.

In one or more exemplary drug delivery devices, the actuator mechanism is configured to move, e.g. by rotation about a second rotation axis of the second attachment part (second base part), the second distal end from a second primary angular position of second primary position to a second secondary angular position of second secondary position in relation to a second proximal end of the second attachment part. An angle between the second primary angular position and the second secondary angular position may be larger than 10°, such as larger than 45° or larger than 60°.

In one or more exemplary drug delivery devices, the drug delivery device can include a locking system. The locking system can be configured to prevent the actuator mechanism from converting the stored energy to the kinetic energy. For example, the locking system may be configured to lock, e.g. prevent rotation of, the first body part in relation to the second body part in a first state of the drug delivery device. The locking system may be configured to lock the first attachment part in a first primary position, e.g. in relation to the first body part, when the drug delivery device is in the first state. Upon release of the locking system, the first attachment part may be allowed to move from a first primary position to a first secondary position, e.g. through the conversion of stored energy to kinetic energy. The locking system may upon release be configured to allow rotation of the first body part in relation to the second body part, e.g. in a second state of the drug delivery device. The locking system may be configured to lock the second attachment part in a second primary position, e.g. in relation to the second body part, when the drug delivery device is in the first state. The locking system may upon release be configured to allow the second attachment part to move from a second primary position to a second secondary position.

In one or more example drug delivery devices, the drug delivery device can include an aperture, such as a whole, gap, slot, cavity. In one or more example drug delivery devices, the drug delivery device can include a plurality of apertures. The aperture can be in the first body part. The aperture can be in the second body part. The aperture can be in both the first body part and the second body part. For example, the aperture can span between the first body part and the second body part. The aperture can extend across both the first body part and the second body part.

The aperture may be on an outer surface of the first body part and/or the second body part. The aperture may face outward from the first body part and/or the second body part. The aperture may extend inward into the first body part and/or the second body part.

The aperture may narrow from an outer portion of the first body part and/or the second body part to an inner portion. A radially outwardmost portion of the aperture may have larger dimensions than a radially inwardmost portion. For example, a cross section of the aperture taken along the central axis may be greater at a radially outwardmost portion of the aperture than a radially inwardmost portion. The aperture may be considered part of the locking system. The aperture may be considered not part of the locking system.

The locking system may include a locking member, such as a locking plug, locking insert. The locking system may include a plurality of locking members. The locking system may include as many locking members as apertures. Alternatively, a single locking member may be used for a plurality of apertures.

The locking member may be configured to be releasably retained in the aperture. The locking member may be configured to be inserted into the aperture. The locking member may be configured to mate within the aperture. The locking member may be configured to fit within the aperture. An outer surface of the locking member may be configured to fit within surfaces formed by the aperture. The locking member may form a flush surface when inserted into the aperture. The locking member may protrude from the first body part and/or the second body part when inserted. The locking member may span both the first body part and the second body part. The locking member may only be located in the first body part. The locking member may only be located in the second body part.

The locking member may have a particular shape. For example, the locking member may be pyramid shaped. The locking member may be diamond shaped. The locking member may be ball shaped. The locking member may be cone shaped. The locking member may be wedge shaped. The locking member may be H-shaped. The locking member may be Z-shaped. The locking member may be I-shaped. The locking member may be butterfly-shaped. The locking member may be a combination of one or more of a diamond shape, a ball shape, a cone shape, a wedge shape, an H shape, a Z shape, and an I shape.

Depth of the locking member, for example into the aperture, can be adjustable and/or controllable. For example, the depth of the locking member may be adjusted to control actuation of the actuator element.

The locking member may be a shape that, upon receiving a force, is configured to translate. The locking member may be a shape that, upon receiving a compressive force, is configured to translate. For example, the locking member can be configured to translate out of the aperture, such as away from the aperture. The locking member may be a shape that, upon receiving a rotational force, is configured to translate. In one or more example drug delivery devices, converting the stored energy to the kinetic energy is configured to translate the locking member out of the aperture. For example, the locking member may be self-ejecting.

The locking member may narrow from a radially outward direction to a radially inward direction. A radially outwardmost portion of the locking member may have larger dimensions than a radially inwardmost portion. If a force is applied on the narrower portion of the locking member, it can be forced out of the aperture.

When inserted, such as located, in the aperture and retained in place, the locking member may prevent the conversion of the stored energy to the kinetic energy. For example, the locking member can prevent rotation of the first body part with respect to the second body part. When the locking member is removed, the conversion of the stored energy to the kinetic energy can occur. For example, the first body part can rotate with respect to the second body part. In one or more example drug delivery systems, when the locking member is inserted into the aperture and not retained in place, it may not prevent the conversion of the stored energy to the kinetic energy. For example, the actuator mechanism may begin to convert the stored energy to the kinetic energy, thereby translating the locking member out of the aperture, which allows the actuator mechanism to continue converting stored energy to the kinetic energy until the locking member is completely out of the aperture.

The particular shape of the locking member is not limiting, but conversion of the stored energy to kinetic energy, such as causing rotation of the first body part with respect to the second body part, may translate the locking member out of the aperture. For example, the locking member can translate out of the aperture orthogonal to the central axis. The locking member may not translate out of the aperture parallel to the central axis. In one or more example drug delivery devices, the locking member may have a shaped configured to be forced out, such as translated out, of the aperture upon compression of the locking member.

In one or more example drug delivery devices, the locking member can be depositioned or repositioned, such as switching, changing, moving, rotating, translating, converting between an on configuration, or position, and an off configuration, or position. The on configuration, or position, can prevent the conversion of stored energy to kinetic energy. The off configuration, or position, can allow for conversion of the stored energy to kinetic energy. The locking member may not translate away from the drug delivery device, but instead may stay attached to the first body part and/or the second body part in both the on configuration, or position, and the off configuration, or position. Degradation of the cover element, such as discussed herein, may allow the locking member to switch between the on configuration, or position, and the off configuration, or position.

For example, at least a portion of the locking member may be located in the aperture when the biodegradable cover is un-degraded. Upon degradation of the biodegradable cover, the locking member may translate out of the aperture while still remaining attached to the drug delivery device, such as the first part and/or the second part, allowing conversion of the stored energy to kinetic energy.

In one or more example drug delivery devices, the locking member is not biodegradable. The locking member can be partially biodegradable. The locking member may have sections, such as portions or components, that are biodegradable.

The locking system may include an additional component to prevent the locking member from translating out of the aperture. For example, a biodegradable cover can be used to prevent translation of the locking member out of the aperture. The biodegradable cover may be configured to retain, such as hold, the locking member in the aperture. Without the biodegradable cover, the locking member could be forced out of the aperture, such as via the conversion of energy from the actuator mechanism. A plurality of biodegradable covers can be used as well.

As an example, the locking member can be inserted into the aperture. Without any further component of the locking system, the actuator mechanism would convert stored energy to kinetic energy, such as via rotation of the first body part with respect to the second body part, which would translate the locking member out of the aperture. However, the biodegradable cover can prevent this translation, thereby preventing the actuator mechanism from converting of the stored energy to kinetic energy, which can prevent rotation of the first body part with respect to the second body part.

The biodegradable cover can be attached to the first body part and/or the second body part. The biodegradable cover can surround the first body part and/or the second body part. The biodegradable cover can mate with the first body part and/or the second body part. The biodegradable cover can be located on an outer surface of the first body part and/or the second body part. The biodegradable cover can be located on a radially outermost surface of the first body part and/or the second body part.

The biodegradable cover can partially cover the locking member. The biodegradable cover can fully cover the locking member. The biodegradable cover may cover a plurality of locking members.

In one or more example drug delivery devices, an entirety of the biodegradable cover is biodegradable. In one or more example drug delivery devices, a portion less than an entirety of the biodegradable cover is biodegradable. In one or more example drug delivery devices, the biodegradable cover can include portions, such as sections, that are biodegradable and portions, such as sections, that are not biodegradable.

In one or more example drug delivery devices, the biodegradable cover can include a biodegradable section. Degradation of the biodegradable section can release the biodegradable cover from the first body part and/or the second body part. Release of the biodegradable cover can allow for translation of the locking member from the aperture.

The biodegradable cover can be biodegradable. The biodegradable cover may be dissolvable. The biodegradable cover may be configured to degrade. The biodegradable cover may be configured to lose integrity. The biodegradable cover may be configured to lose strength. For example, as the biodegradable cover is hydrated, the biodegradable cover may lose integrity.

In one or more exemplary drug delivery devices, the biodegradable cover may be made of a biodegradable material. The biodegradable cover can include an absorbable material. The biodegradable cover can be a material which loses coherence upon oral dosing. The biodegradable cover may be configured to degrade. The biodegradable cover may be configured to lose integrity. The biodegradable cover may be configured to lose strength. For example, the biodegradable cover may be configured to lose strength or lose integrity from hydration.

The biodegradable cover can include a material which allows the material of the attachment part to be broken down, degraded and/or dissolved by processes that are present in the body, such as corrosion, degradation, hydrolysis and/or proteolytic enzymatic degradation. Thus, when biodegradable cover has been inside the human body for a period of time, the biodegradable cover may dissolve, decompose, or degrade to such a degree that the biodegradable cover may lose its structural stability. This may allow for the translation of the locking member out of the aperture, which can then allow the actuator mechanism to convert the stored energy to the kinetic energy. In one or more example drug delivery devices, upon weakening of the biodegradable cover, the actuator mechanism is configured to convert the energy to the kinetic energy and translate the locking member out of the aperture.

In one or more example drug delivery devices, the biodegradable cover may degrade in a particular pH, while not degrading in other pHs. For example, the biodegradable cover may be configured to not degrade in an oral cavity. This can include, for example, a pH of 7.0 or greater. The biodegradable cover may be configured to degrade in the stomach. The biodegradable cover may be configured to degrade in the intestines. Thus, the biodegradable cover may be configured to degrade at a pH below 7.0, below 6.0, below 5.5, below 5.0, below 4.5, below 4.0, below 3.5, below 3.0, below 2.5, below 2.0, below 1.5, or below 1.0.

The biodegradable cover can be may be configured to dissolve when the drug delivery device enters the gastrointestinal tract or at a desired location in the gastrointestinal tract, thereby releasing the first body part in relation to the second body part and allowing the actuator mechanism to convert stored energy to kinetic energy to rotate the first body part in relation to the second body part and thereby moving the first distal end towards the second distal end in turn resulting in attachment of the drug delivery device to the internal tissue.

Upon degradation, which can include either partial degradation or full degradation, of the biodegradable cover, the locking member can translate out of the aperture.

Upon degradation, which can include either partial degradation or full degradation, of the biodegradable cover, the actuator mechanism may be configured to convert the stored energy to kinetic energy, thus translating the locking member out of the aperture.

Accordingly, the locking system can be optionally configured to lock and/or unlock (release) the first body part in relation to the second body part. The locking system can be configured to lock and/or unlock (release) the first attachment part in relation to the first body part. The locking system can be configured to lock and/or unlock (release) the second attachment part in relation to the second body part.

The biodegradable cover may cover a portion of the drug delivery device. In one or more example drug delivery devices, the biodegradable cover can surround an entirety of the drug delivery device. For example, the biodegradable cover may be a capsule. The biodegradable cover may be a biodegradable capsule. The biodegradable cover may cover, such as surround, the locking member.

In one or more example drug delivery devices, the biodegradable cover can surround a portion of the drug delivery device. In one or more example drug delivery devices, the biodegradable cover may not cover the attachment part.

The biodegradable cover can be a shrink wrap. The biodegradable cover may be a coating. The biodegradable cover may be a film.

The biodegradable cover may be, for example, a ring, loop, partial ring, or partial loop. The biodegradable cover may have a circumferential length greater than a longitudinal width. For example, the circumferential length may be 2×, 3×, 4×, 5×, 6×, 7×, 8×, 9×, or 10× the longitudinal width. The biodegradable cover may fit on an outer surface of the drug delivery device. For example, the biodegradable cover may be located on an outer surface of the first body part or the second body part. The biodegradable cover may be mechanically fit onto the drug delivery device. For example, the biodegradable cover may be snap fit onto the drug delivery device. The biodegradable cover may be chemically attached onto the drug delivery device.

In one or more exemplary drug delivery devices, the biodegradable cover could be in the shape of a capsule part. The biodegradable cover may be a capsule. The biodegradable cover may be a single capsule. The biodegradable capsule may form two capsule parts. For example, the biodegradable cover could form a first half of a capsule. The biodegradable cover could form a first half of a capsule and a second biodegradable cover could form a second half of the capsule. When fitted together, the biodegradable cover and the second biodegradable cover could form a full capsule. In one or more example drug delivery devices, the biodegradable cover can include a first section in connection with a second section. When combined, the first section and the second section can form a capsule.

The biodegradable cover may partially or fully cover the first body recess if located on the first body. Thus, the biodegradable cover may prevent motion of the first attachment part. The biodegradable cover may partially or fully cover the second body recess if located on the second body. Thus, the biodegradable cover may prevent motion of the second attachment part. The biodegradable cover may partially or fully cover both the first body recess and the second body recess. The biodegradable cover may partially or fully cover the first body recess and a second biodegradable cover may partially or fully cover the second body recess.

The biodegradable cover may extend fully along an outer circumference of the drug delivery device. The biodegradable cover may extend around 10, 15, 20, 25, 30, 35, 40, 45, 50, 55, 60, 65, 70, 75, 80, 85, 90, 95, or 100% of an outer circumference of the drug delivery device. The biodegradable cover may extend around greater than 10, 15, 20, 25, 30, 35, 40, 45, 50, 55, 60, 65, 70, 75, 80, 85, 90, or 95% of an outer circumference of the drug delivery device. The biodegradable cover may extend around less than 10, 15, 20, 25, 30, 35, 40, 45, 50, 55, 60, 65, 70, 75, 80, 85, 90, 95, or 100% of an outer circumference of the drug delivery device.

In one or more example drug delivery devices, the locking member can be held within the aperture. This can prevent the conversion of stored energy to kinetic energy. While the locking member is held within the aperture, the biodegradable cover can be placed on the drug delivery device. For example, the drug delivery device can be dipped into a liquid which can form the biodegradable cover. Alternatively, the first section of the biodegradable capsule can be placed on the drug delivery device. The second section of the biodegradable capsule can then be placed on the drug delivery device. In one or more example embodiments, the locking member can be released after placing the first section on the drug delivery device if the first section is sufficient to hold the locking member in place. Alternatively, the entirety of the biodegradable capsule can be placed on the drug delivery device at once.

In one or more exemplary drug delivery devices, at least part of the first attachment part and/or the second attachment part may be made of a biodegradable material, absorbable material, or similar material which allows the material of the attachment part to be broken down, degraded and/or dissolved by processes that are present in the body, such as corrosion, degradation, hydrolysis and/or proteolytic enzymatic degradation. Thus, when the attachment part(s) has been inside the human body for a period of time, the attachment part(s) may dissolve, decompose or degrade to such a degree that the attachment part may lose its structural stability, which may in turn release the drug delivery device from the surface it has attached itself to. Thus, after a period, e.g. when the drug substance has been released from the attachment part(s), the attachment part(s) may deteriorate to such a degree that the drug delivery device may be released and may continue its journey through the gastrointestinal tract to be released through natural intestinal and/or bowel movements of the user or patient.

In one or more exemplary drug delivery devices, the rotational axis of the first body part and/or the second body part (primary axis) may be the central axis of the drug delivery device, e.g. the primary axis of the first body part may be coaxial to the central axis. Thus, the central axis intersects both the first body part and the second body part, and may define the primary axis.

In one or more exemplary drug delivery devices, the first body part and the second body part may be substantially symmetrical in a radial direction perpendicular to the central axis. This may mean that the first body part and/or the second body part may have a circular periphery, where the periphery may extend in a radial direction away from and perpendicular to the central axis.

The first attachment axis may be seen as an axis that is coaxial with the length of the first attachment part. The second attachment axis may be seen as an axis that is coaxial with the length of the second attachment part. In case the first attachment part has a shape that is not straight, the first attachment axis may be defined as an axis that intersects the first distal end and the first proximal end of the first attachment part. In case the second attachment part has a shape that is not straight, the second attachment axis may be defined as an axis that intersects the second distal end and the second proximal end of the second attachment part.

In one or more exemplary drug delivery devices, the first attachment axis may be positioned at a first distance from the central axis, while the second attachment axis may be positioned at a second distance from the central axis and/or primary axis.

For example, the first attachment axis may be positioned at a first primary distance from the central axis in the first state of the drug delivery device. The first primary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The first attachment axis may cross or be close to (distance less than 0.5 mm) the central axis in the first state of the drug delivery device.

The first attachment axis may be positioned at a first secondary distance from the central axis in the second state of the drug delivery device. The first secondary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The first attachment axis may cross or be close to (distance less than 0.5 mm) the central axis in the second state of the drug delivery device.

For example, the second attachment axis may be positioned at a second primary distance from the central axis in the first state of the drug delivery device. The second primary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The second attachment axis may cross or be close to (distance less than 0.5 mm) the central axis in the first state of the drug delivery device.

The second attachment axis may be positioned at a second secondary distance from the central axis in the second state of the drug delivery device. The second secondary distance may be larger than 0.5 mm, such as in the range from 1 mm to 15 mm or larger than 1 mm, e.g. 2 mm, 3 mm, 4 mm, 5 mm, 6 mm, 7 mm, 8 mm, 9 mm, 10 mm, 11 mm, 12 mm, 13 mm, or 14 mm.

The second attachment axis may cross or be close to (distance less than 0.5 mm) the central axis in the second state of the drug delivery device.

In one or more exemplary drug delivery devices, the first attachment part (first attachment axis) and/or the second attachment part (second attachment axis) may be configured to be at an angle to each other when they intersect a plane that includes the central axis. The plane may be a plane that contains the central axis, where the plane also includes a radial axis extending at a right angle from the central axis. When the first attachment part intersects the plane, the first attachment axis of the first attachment part may be at an angle to the plane so that the first distal end of the first attachment part is the first part of the first attachment part that intersects the plane, where the remaining parts of the first attachment part intersects the plane subsequently during rotational movement. The second attachment part may intersect the same plane from the opposite side, where the second distal end of the second attachment part is the first part of the second attachment part that intersects the plane, where the remaining parts of the second attachment part intersects the plane subsequently during rotational movement. Thus, the second attachment part optionally intersects the plane from an opposing rotational direction than the first attachment part. This may also mean that when the first distal end and the second distal end of respective first attachment part and second attachment parts each contact the plane, the first attachment part (first attachment axis) is at an angle to the plane as well as at an angle to the second attachment part (second attachment axis). The angle between the first attachment part (first attachment axis) and the second attachment part (second attachment axis) to the plane may be approximately half the size of the angle between the first attachment part and the second attachment part.

In one or more exemplary drug delivery devices, the first body part may be configured to rotate in a first direction and the second body part may be configured rotate in a second direction, where the first direction is opposite to the second direction. Thus, as an example, the first body part may rotate in a clockwise direction, while the second body part may rotate in an opposed anti clockwise direction. In one or more examples where the drug delivery device comprises three or more body parts, abutting or neighbouring body parts may rotate in opposite directions. This may also mean that every second body part may rotate in the same direction. For example, where a first body part and a third body part rotate in the same first direction, then a second body part and/or a fourth body part may rotate in a second direction opposite the first direction.

In one or more exemplary drug delivery devices, the actuator mechanism may comprise one or more resilient parts, such as a plurality of resilient parts. The resilient parts can be configured to store energy. The resilient parts can be configured to convert the stored energy to kinetic energy.

In one or more exemplary drug delivery devices, the first distal end of the first attachment part and/or the second distal end of the second attachment part may be provided with a sharp tip configured to penetrate a biological tissue. The sharp tip may be positioned in the vicinity of the distal end of the respective attachment part, where the sharp tip may be configured to have a diameter at the distal end which is smaller than a diameter of the attachment part at a distance from the distal end. The sharp tip may be configured in such a manner that when a rotational force is applied to the first attachment part, and a counterforce is applied to the second attachment part, the counterforce may cause the sharp tip to penetrate the biological tissue due to the force applied by the actuator mechanism.

When the first attachment part and/or the second attachment part penetrates the biological tissue due to the rotation between the first body part and the second body part (the first distal end moving towards the second distal end, the respective penetration point(s) in the biological tissue may be utilized to deliver a drug substance from the drug delivery device into the biological tissue, and where the drug substance may be introduced into the biological tissue that is beyond the mucous membrane. Thereby, the drug substance may enter the bloodstream more easily than if the drug substance is released in the stomach or intestinal lumen, and the drug delivery may be more effective. An example of this is when the drug substance is insulin, where insulin may degrade inside the gastrointestinal tract and is not capable of being absorbed from the gastrointestinal tract, but where a mucous membrane has been penetrated, and the insulin released through the penetrated gastrointestinal wall, the insulin will remain intact and reach the bloodstream of the user via the blood vessels in the intestinal layer beyond the mucous membrane (surface).

In one or more exemplary drug delivery devices, the first attachment part and/or the second attachment part may be provided with a gripping part configured to grip a biological tissue. The gripping part may be utilized to improve traction between the attachment part and a mucous membrane, allowing the attachment part to anchor the drug delivery device inside the body of the user. The gripping part may be a part that increases a mechanical friction between the attachment part and the surface to be attached to, where the gripping part may e.g. have a hook shape, or e.g. a shape where the gripping part of the first attachment part faces the gripping part of the second attachment part, so that the biological tissue which is positioned between the first attachment part and the second attachment part is gripped between the two gripping parts.

In one or more exemplary drug delivery devices, a part of the resilient part may be connected to the first body part and a second part of the resilient part may be connected to the second body part. This means that the resilient part may be utilized to store energy, such as rotational energy, elastic potential energy, or rotational force which is applied to the first body part and the second body part, where the energy is stored in the resilient part. Furthermore, when the energy is released, e.g. when a locking element is dissolved or degraded, e.g. when the stored energy is converted into kinetic energy, the force may be released to both the first body part and the second body part, which in turn transfers the force to the first attachment part and the second attachment part. The resilient part may e.g. be in the form of a helical spiral spring (mainspring) and/or a spiral torsion spring, where the first body part may be wound relative to the second body part by rotating the first body part relative to the second body part. This stores energy in the mainspring by twisting the spiral tighter. The stored force of the mainspring may then rotate the first body part in the opposing direction as the mainspring unwinds. Thus, the force of the mainspring may cause the first attachment part and the second attachment parts to travel in opposing directions, and where the attachment parts may pinch the biological tissue and either pinch the tissue or penetrate the tissue in order to attach the drug delivery device to the biological tissue.

When the drug delivery device has entered the body, and has e.g. entered the desired part in the gastrointestinal tract, the drug delivery device may be configured to transform from the first state to the second state. The transformation may be initiated by different means, where e.g. the first and the second body parts may be held in the first state using the biodegradable cover made of a dissolvable, expandable or degradable material, where the material reacts with the surroundings, such as fluids, inside the desired body part, thereby unlocking or releasing the locking mechanism. The material of the biodegradable cover may be a material that loses its structural force when in contact with the surroundings inside the desired body part. An example may be where biodegradable cover is made of a polymeric material or a sugary substance which may dissolve, expand, or degrade when it comes into contact with a certain kind of fluid which may include an enzyme or a certain kind of acid inside the digestive system. When the biodegradable cover comes into contact with the reagent, the material may dissolve, expand or degrade over time, and when the rotational force of the drug delivery device exceeds the static force of the locking element, the rotational force may be released via a rotation of the first body part relative to the second body part, or vice versa.

In one or more exemplary drug delivery devices, a biodegradable cover may fix an attachment part in a position where the attachment element locks the first body part in relation to the second part, i.e. prevents the first body part from rotating in relation to the second body part. When the biodegradable cover dissolves or degrades, the attachment part can move to a secondary position where the attachment part does not lock the first body part in relation to the second part, e.g. by the actuator mechanism causing a rotation of the attachment part about a rotation axis in relation to the body part to which the attachment part is rotationally attached.

The second state of the drug delivery device may be seen as the state which is initiated by the release of energy stored, e.g. conversion of the stored energy into kinetic energy, in the actuator mechanism, e.g. resilient part(s) of the actuator mechanism into a rotational force of the first and/or the second body part and/or a rotational force of the first attachment part in relation to the first body part. A termination of the second state may be seen as a point in time where the energy stored in the resilient part becomes stationary again, i.e. when the attachment parts have gripped or penetrated biological tissue and/or the rotational movement between the first body part and the second body part is stopped.

In one or more exemplary drug delivery devices, the drug delivery device may have a first state where the actuator mechanism has a constant resilient force load and a second state where the actuator mechanism releases the resilient force load, e.g. converts the stored energy to kinetic energy. In the first state, the constant resilient force load may be seen as the energy stored in the actuator mechanism, and where the resilient force load is larger than zero. The second state may be seen as a state where the actuator mechanism releases its resilient force load, where the resilient force load is reduced, e.g. approaches zero, e.g. by rotating the first body part in relation to the second body part. The second state may be terminated when the attachment parts come into contact with or penetrates a biological tissue and the resilient force load does not change, even though it has not reached zero. Thus, a third state may follow the second state, when the drug delivery device has been attached to a wall of biological material, and the resilient force load is stationary after a resilient force release.

The first attachment part and/or the second attachment part may have an unfolding function, where during the first state of the drug delivery device, i.e. the initial state of the drug delivery device, the attachment parts are positioned or arranged inside the first and/or the second body part, or alternatively where the first and/or second attachment parts may be folded along the sides of the body parts. Other ways of obtaining the same may be envisioned. The folded state (first state) may e.g. be maintained using a releasable locking mechanism in the form of an encapsulation similar to a drug substance capsule, a band or plug, e.g. made of gelatine, sugars or other dissolvable materials or materials that lose their structural force. For example, this can include the biodegradable cover. Thus, the attachment parts may be held in place until the drug delivery device has entered the gastrointestinal tract, e.g. the stomach, so that the attachment parts do not interfere or damage the lining of the mouth and/or the oesophagus. Prior to or during the transition to the second state the attachment parts may extend from the body parts and outwards, making the attachment parts ready to interact with a lining of the digestive system. When the attachment part or parts are in a folded or collapsed position, the distance from the central axis to the distal end of the attachment part being longer in the second state than in a first state. Thus, the diameter of the drug delivery device in the first state will be less in than the diameter of the drug delivery device in the second state.

In one or more exemplary drug delivery devices, at least part of the first attachment part and/or the second attachment part, such as the first needle and/or the second needle may be made of material comprising one or more of magnesium, titanium, iron and zinc which allows for accurate and precise control of the size and/or shape/geometry of the first attachment part and/or second attachment part in turn allowing for a delivery device with desired attachment capabilities and/or small production variances which is in particular important in the pharmaceutical industry.

The first attachment part, such as the first needle, may be made of material comprising one or more of magnesium, titanium, iron and zinc. The material of the first attachment part/first needle may be biocompatible and/or biodegradable such as a biocompatible material and/or a biodegradable material. The material of the first attachment part/first needle may comprise one or more biodegradable polymers such as PLA and/or POLGA. Some of, part of, most of, substantially all, or all of the material of the first attachment part/first needle may be biocompatible and/or biodegradable. The material of the first attachment part, such as the first needle, may comprise, consist of, or essentially consist of, biocompatible and/or biodegradable material such as biocompatible and/or biodegradable metals. The material of the first attachment part, such as the first needle, may comprise a biodegradable or bioresorbable metal or metal alloy, such as magnesium, zinc, and/or iron or an alloy comprising one or more of magnesium, zinc and iron. A biodegradable or bioresorbable metal or metal alloy may be understood as a metal or metal alloy that degrades safely within e.g. a human body in a practical amount of time, for example related to their application. The material of the first attachment part, such as the first needle, may comprise one or more metals such as a combination of one or more metals e.g. as a metal alloy.

The second attachment part, such as the second needle, may be made of material comprising one or more of magnesium, titanium, iron and zinc. The material of the second attachment part/second needle may be biocompatible and/or biodegradable such as a biocompatible material and/or a biodegradable material. The material of the second attachment part/second needle may comprise one or more biodegradable polymers such as PLA and/or POLGA. Some of, part of, most of, substantially all, or all of the material of the second attachment part/second needle may be biocompatible and/or biodegradable. The material of the second attachment part, such as the second needle, may comprise, consist of, or essentially consist of, biocompatible and/or biodegradable material such as biocompatible and/or biodegradable metals. The material of the second attachment part, such as the second needle, may comprise a biodegradable or bioresorbable metal or metal alloy, such as magnesium, zinc, and/or iron or an alloy comprising one or more of magnesium, zinc and iron. A biodegradable or bioresorbable metal or metal alloy may be understood as a metal or metal alloy that degrades safely within e.g. a human body in a practical amount of time, for example related to their application. The material of the second attachment part, such as the second needle, may comprise one or more metals such as a combination of one or more metals e.g. as a metal alloy.

An advantage of having a biodegradable material used in the attachment part(s) may be that the delivery device is able to deliver an active drug substance or payload arranged in the attachment part(s) and/or body parts of the delivery device at a specific part of the body of the subject, e.g. such as the stomach or intestines after the delivery device has attached to the internal surface, e.g. the intestinal wall, thanks to the sharp properties of the material of the attachment part(s), and for an extended period of time, since the biodegradable material will degrade gradually in time. Further, when the material of the attachment part(s) is biodegradable, the attachment part(s) will degrade in the human body and disappear after having delivered the payload/active drug substance comprised in the drug delivery device, thereby avoiding harming the human subject over time. The attachment part(s) may be configured to degrade in a period of time of hours, e.g. 2 hours, 5 hours, 10 hours, 20 hours, or 24 hours, days, e.g. 1 day, 2 days, 5 days, or weeks, e.g. 1 week, 2 weeks, 3 weeks, or 5 weeks.

The material of the attachment part(s), such as the needle(s), may comprise one or more or a combination of magnesium (Mg), zinc (Zn), and/or iron (Fe). An advantage of having the attachment part(s) of a material comprising Mg, Zn, and/or Fe may be that the shape and size of the attachment part(s) can be precisely controlled thereby providing improved attachment to the internal surface, for example to an internal wall of the intestines of the human subject.

The attachment part(s), such as the needle(s), may be made of a material comprising one or more thermoplastic or thermoset polymers. The material of the attachment part(s), such as the needle(s), may comprise one or more active drug substances. Thus, an active drug substance may be embedded in the material of the attachment part(s), such as the needle(s), to form a pharmaceutical composition.

In some embodiments, the attachment part(s), such as the needle(s), may comprise for example water soluble, water insoluble, biodegradable, non-biodegradable and/or pH dependent soluble materials. In some embodiments, the attachment part(s), such as the needle(s), may comprise a water soluble, biodegradable and/or pH-dependent material that may dissolve and/or degrade so that the attachment part(s), such as the needle(s), lodged in the intestinal tissue may gradually degrade and/or dissolve. In some embodiments, the attachment part(s), such as the needle(s), may comprise a water-soluble material to allow immediate release or modified release of the active drug substance depending of the material selected. In some embodiments, a water insoluble or biodegradable material may allow depot of the active drug substance in the attachment part(s), such as the needle(s), for longer release duration (for example days, weeks or months). In some embodiments, a pH dependent soluble material may allow the attachment part(s), such as the needle(s), to stay intact at pH conditions below the physiologic for example a pH of approximately 7.4 to remain intact in the gastrointestinal lumen, but then may dissolve once inside the gastrointestinal wall. In some embodiments, one or more water soluble, water insoluble, biodegradable and/or pH dependent materials may optionally be combined to control release of the active drug substance for example by diffusion or erosion of the attachment part(s), such as the needle(s), for controlled release duration (for example minutes, hours, days, weeks, or months).

In some embodiments, the attachment part(s), such as the needle(s), may be made from different compositions. For example, an outer part of the attachment part(s), such as the needle(s), may be made of one composition and an inner core of the attachment part(s), such as the needle(s), may be made from another composition. In some embodiments, the outer part and the inner core of the attachment part(s), such as the needle(s), may be composed of for example a water soluble, a water insoluble, a biodegradable, and/or a pH dependent material. In some embodiments, one or more water soluble, water insoluble, biodegradable and/or pH dependent materials may be combined to control the release of the active drug substance once the attachment part(s), such as the needle(s), may move its position from the lumen to the internal tissue for example the gastrointestinal lumen to the gastrointestinal tissue.

In some embodiments, the attachment part(s), such as the needle(s), may be tubular and may include a tubular body and the tubular body may comprise an active drug substance for example a liquid payload comprising the active drug substance, optionally connected to a tubular attachment part so the payload with the active drug substance may flow though the attachment part(s), such as the needle(s), into the internal tissue for example the intestinal tissue. In some embodiments, the tubular body may contain expandable such as swelling excipients that may expand by a chemical reaction for example when mixed expand in volume and/or produce a gas to advance the delivery of the payload. In some embodiments, the expansion is by osmosis.

In some embodiments, the first compartment (compartment to hold active drug substance) may comprise a closure part for closing the first compartment. The closure part may contribute to improved control of release of the active drug substance. In some embodiments, the closure part may be composed of for example a water soluble, a water insoluble, a biodegradable, and/or a pH dependent material. In some embodiments, one or more water soluble, water insoluble, biodegradable and/or pH dependent materials may be combined to control the release of the active drug substance from the first compartment once the attachment part(s), such as the needle(s), moves its position from the lumen to the internal tissue for example from the gastrointestinal lumen to the gastrointestinal tissue.

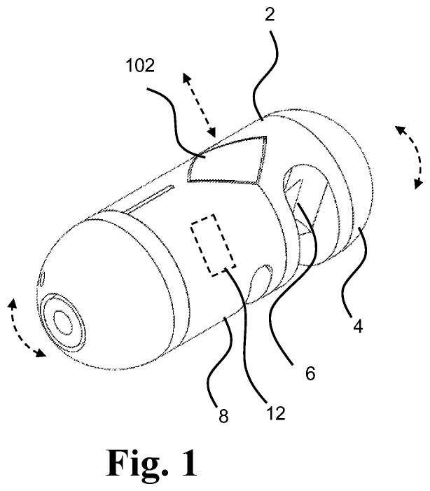

FIG. 1 illustrates a diagram of an example drug delivery device 2 according to the disclosure.

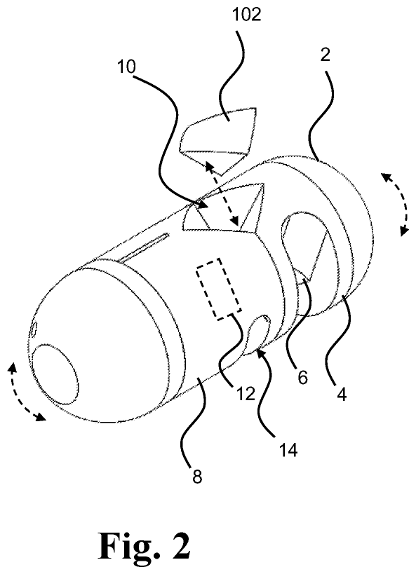

As shown, the drug delivery device 2 can have a first body part 4 and an attachment part 6 attached to the first body part 4. Further, the drug delivery device 2 can include a second body part 8. The second body part 8 may be associated with the first body part 4. The first body part 4 may be rotatable with respect to the second body part 8. The first body part 4 may be translatable with respect to the second body part 8. Optionally, the drug delivery device 2 may have a second attachment part 14 (shown in FIG. 2) attached to the second body part 8.

As shown in FIG. 2, the drug delivery device 2 can include an aperture 10. The aperture can be in the first body part 4 and/or the second body part 8. As shown in FIG. 2, the aperture 10 spans both the first body part 4 and the second body part 8. A locking member 102 can be configured to be releasably retained in the aperture 10.

The drug delivery device 2 can further include an actuator mechanism 12. The actuator mechanism 12 can be located internal of the drug delivery device 2. The actuator mechanism 12 can be configured to store energy and convert the stored energy to kinetic energy. For example, the actuator mechanism 12 may be a spring, and thus can store elastic potential energy. The actuator mechanism 12 can convert the stored energy to kinetic energy via movement of the first body part 4 or the second body part 4. For example, the actuator mechanism can rotate the first body part 4 with respect to the second body part 4, or vice versa. The actuator mechanism 12 can be configured to convert the stored energy to the kinetic energy by rotating at least one of the first body part 4 or the second body part 8 with respect to one another.

This rotation can allow for translation of the locking member 102 out of the aperture 10, as shown in FIGS. 1-2. Specifically, converting the stored energy to the kinetic energy is configured to translate the locking member 102 out of the aperture 10. Further, the conversion can rotate the attachment part 6 in order to pierce tissue.

The shape of the locking member 102 can allow for the translation out of the aperture 10. For example, the locking member 102 can be one or more of pyramid shaped, diamond shaped, ball shaped, cone shaped, or wedge shaped. The locking member 102 can be compressed, such as squeezed, out of the aperture 10.

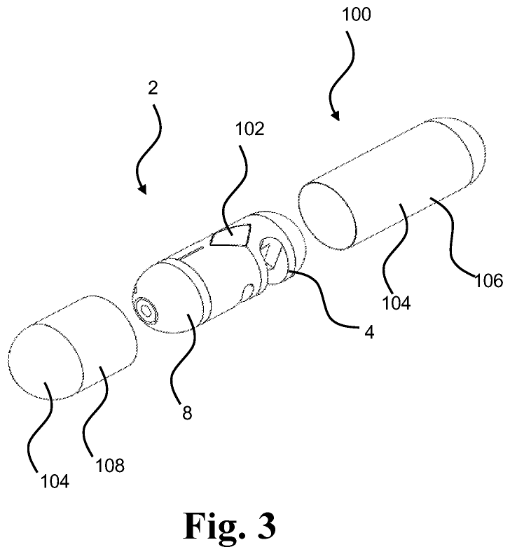

FIG. 3 shows a diagram of an example drug delivery device 2 according to the disclosure. A shown, the drug delivery device 2 can include a locking system 100. The locking system 100 can be configured to prevent the actuator mechanism 12 from converting the stored energy to the kinetic energy. The locking system 100 can include the locking member 102. The locking member 102 can be configured to be releasably retained in the aperture 10 and prevent movement of the first body part 4 with respect to the second body part 8. For example, the locking member 102 can prevent rotational movement.

The locking system 100 can further include a biodegradable cover 104. The biodegradable cover 104 can be configured to prevent translation of the locking member 102 out of the aperture 10. For example, the biodegradable cover 104 can keep the locking member 102 in place, which can prevent the conversion of stored energy to kinetic energy by the actuator mechanism 12.

The locking member 102 can be inserted into the aperture 10 to prevent the conversion of stored energy to the kinetic energy, such as via rotation of the first body part 4 with respect to the second body part 8. However, due to the shape of the locking member 102, the locking member 102 will be forced out of the aperture 10 without the biodegradable cover 104 retaining the locking member 102. The biodegradable cover 104 can prevent the translation until an appropriate time when the biodegradable cover 104, for example, has been degraded or weakened. For example, upon weakening of the biodegradable cover 104, the actuator mechanism 12 is configured to convert the energy to the kinetic energy and translate the locking member 102 out of the aperture 10.

While the biodegradable cover 104 is biodegradable, the locking member 102 may not be biodegradable.

Further, as shown in FIG. 3, the biodegradable cover 104 may include a first section 106 in connection with a second section 108. This allows a full capsule to be formed by connecting the first section 106 to the second section 108. Thus, the biodegradable cover 104 may be a capsule. Alternatively, the biodegradable cover 104 may be a single piece capsule.

The entirety of the biodegradable cover 104 may be biodegradable. Portions of the biodegradable cover 104 may be biodegradable. The biodegradable cover 104 can include a biodegradable section, and wherein degradation of the biodegradable section, such as first section 106, releases the biodegradable cover 104 from the first body part 4 and/or the second body part 8.

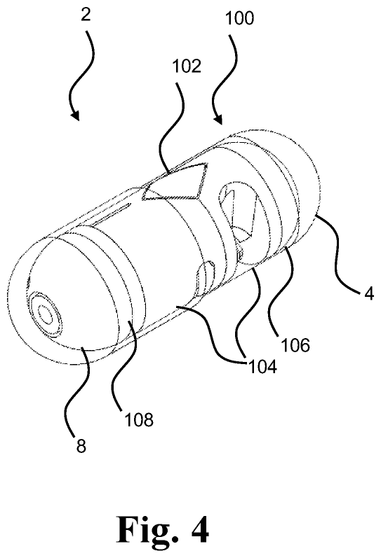

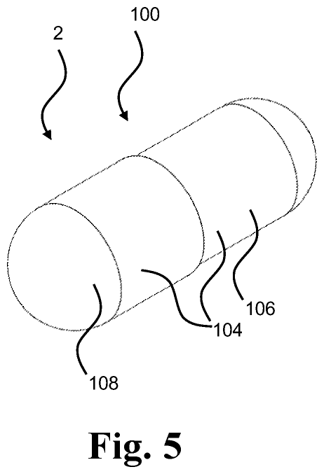

FIG. 4 and FIG. 5 illustrate a diagram of an example drug delivery device 2 with a biodegradable cover 104 being a full capsule 104. FIG. 4 includes a see-through biodegradable cover 104 for convenience. As shown, the biodegradable cover 104 surrounds an entirety of the drug delivery device 2. Alternatively, the biodegradable cover 104 may surround a portion of the drug delivery device 2. For example, the biodegradable cover 104 may not cover the attachment part 106.

Advantageously, the locking system 100 can prevent the locking member 102 from translating out of the aperture 10 until the biodegradable cover 104 has been sufficiently weakened, such as biodegraded. This can allow the attachment part 6 to rotate outward at an opportune time for better insertion into tissue for active drug delivery purposes.

Also disclosed are delivery devices, methods, and compositions according to any of the following items.

Item 1. A drug delivery device comprising:
a first body part;
an attachment part attached to the first body part;
a second body part;
an aperture in the first body part and/or the second body part;
an actuator mechanism configured to store energy and convert the stored energy to kinetic energy via movement of the first body part or the second body part; and
a locking system configured to prevent the actuator mechanism from converting the stored energy to the kinetic energy, the locking system comprising:
   a locking member configured to be releasably retained in the aperture and prevent movement of the first body part with respect to the second body part; and
a biodegradable cover configured to prevent translation of the locking member out of the aperture.

Item 2. Drug delivery device of Item 1, wherein converting the stored energy to the kinetic energy is configured to translate the locking member out of the aperture.

Item 3. Drug delivery device of any one of Items 1-2, wherein upon weakening of the biodegradable cover, the actuator mechanism is configured to convert the energy to the kinetic energy and translate the locking member out of the aperture.

Item 4. Drug delivery device of any one of Items 1-3, wherein the locking member is pyramid shaped.

Item 5. Drug delivery device of any one of Items 1-3, wherein the locking member is diamond shaped.

Item 6. Drug delivery device of any one of Items 1-3, wherein the locking member is ball shaped.

Item 7. Drug delivery device of any one of Items 1-3, wherein the locking member is cone shaped.

Item 8. Drug delivery device of any one of Items 1-3, wherein the locking member is wedge shaped.

Item 9. Drug delivery device of any one of the preceding Items, wherein the locking member is not biodegradable.

Item 10. Drug delivery device of any one of the preceding Items, wherein the biodegradable cover surrounds an entirety of the drug delivery device.

Item 11. Drug delivery device of any one of Items 1-9, wherein the biodegradable cover does not cover the attachment part.

Item 12. Drug delivery device of any one of the preceding Items, wherein the biodegradable cover comprises a first section in connection with a second section.

Item 13. Drug delivery device of any one of the preceding Items, wherein an entirety of the biodegradable cover is biodegradable.

Item 14. Drug delivery device of any one of the preceding Items, wherein the biodegradable cover comprises a biodegradable section, and wherein degradation of the biodegradable section releases the biodegradable cover from the first body part and/or the second body part.

Item 15. Drug delivery device of any one of the preceding Items, further comprising a second attachment part attached to the second body part.

Item 16. Drug delivery device of any one of the preceding Items, wherein the actuator mechanism is configured to convert the stored energy to the kinetic energy by rotating at least one of the first body part or the second body part with respect to one another.

Item 17. Drug delivery device of any one of the preceding Items, wherein the biodegradable cover is a biodegradable capsule.

The use of the terms “first”, “second”, “third” and “fourth”, “primary”, “secondary”, “tertiary” etc. does not imply any particular order, but are included to identify individual elements. Moreover, the use of the terms “first”, “second”, “third” and “fourth”, “primary”, “secondary”, “tertiary” etc. does not denote any order or importance, but rather the terms “first”, “second”, “third” and “fourth”, “primary”, “secondary”, “tertiary” etc. are used to distinguish one element from another. Note that the words “first”, “second”, “third” and “fourth”, “primary”, “secondary”, “tertiary” etc. are used here and elsewhere for labelling purposes only and are not intended to denote any specific spatial or temporal ordering.

Furthermore, the labelling of a first element does not imply the presence of a second element and vice versa.

It is to be noted that the word “comprising” does not necessarily exclude the presence of other elements or steps than those listed.

It is to be noted that the words “a” or “an” preceding an element do not exclude the presence of a plurality of such elements.

It should further be noted that any reference signs do not limit the scope of the claims, that the exemplary embodiments may be implemented at least in part by means of both hardware and software, and that several “means”, “units” or “devices” may be represented by the same item of hardware.

Although features have been shown and described, it will be understood that they are not intended to limit the claimed invention, and it will be made obvious to those skilled in the art that various changes and modifications may be made without departing from the spirit and scope of the claimed invention. The specification and drawings are, accordingly to be regarded in an illustrative rather than restrictive sense. The claimed invention is intended to cover all alternatives, modifications, and equivalents.

### LIST OF REFERENCES

2 drug delivery device
4 first body part
6 attachment part
8 second body part
10 aperture
12 actuator mechanism
14 second attachment part
100 locking system
102 locking member
104 biodegradable cover
106 first section
108 second section

1. A drug delivery device comprising:
a first body part;
an attachment part attached to the first body part;
a second body part;
an aperture in the first body part and/or the second body part;
an actuator mechanism configured to store energy and convert the stored energy to kinetic energy via movement of the first body part or the second body part; and
a locking system configured to prevent the actuator mechanism from converting the stored energy to the kinetic energy, the locking system comprising:
a locking member configured to be releasably retained in the aperture and prevent movement of the first body part with respect to the second body part; and
a biodegradable cover configured to prevent translation of the locking member out of the aperture.

2-17. (canceled)

18. The drug delivery device of claim 1, wherein converting the stored energy to the kinetic energy is configured to translate the locking member out of the aperture.

19. The drug delivery device of claim 1, wherein upon weakening of the biodegradable cover, the actuator mechanism is configured to convert the energy to the kinetic energy and translate the locking member out of the aperture.

20. The drug delivery device of claim 1, wherein the locking member is pyramid shaped.

21. The drug delivery device of claim 1, wherein the locking member is diamond shaped.

22. The drug delivery device of claim 1, wherein the locking member is ball shaped.

23. The drug delivery device of claim 1, wherein the locking member is cone shaped.

24. The drug delivery device of claim 1, wherein the locking member is wedge shaped.

25. The drug delivery device of claim 1, wherein the locking member is not biodegradable.

26. The drug delivery device of claim 1, wherein the biodegradable cover surrounds an entirety of the drug delivery device.

27. The drug delivery device of claim 1, wherein the biodegradable cover does not cover the attachment part.

28. The drug delivery device of claim 1, wherein the biodegradable cover comprises a first section in connection with a second section.

29. The drug delivery device of claim 1, wherein an entirety of the biodegradable cover is biodegradable.

30. The drug delivery device of claim 1, wherein the biodegradable cover comprises a biodegradable section, and wherein degradation of the biodegradable section releases the biodegradable cover from the first body part and/or the second body part.

31. The drug delivery device of claim 1, further comprising a second attachment part attached to the second body part.

32. The drug delivery device of claim 1, wherein the actuator mechanism is configured to convert the stored energy to the kinetic energy by rotating at least one of the first body part or the second body part with respect to one another.

33. The drug delivery device of claim 1, wherein the biodegradable cover is a biodegradable capsule.
